(19) United States
(12) Patent Application Publication
(10) Pub. No.: US 2018/0325746 A1
(43) Pub. Date: Nov. 15, 2018

(54) Disposable Absorbent Undergarment Assembly

(71) Applicant: Jamila Newson, Indianapolis, IN (US)

(72) Inventor: Jamila Newson, Indianapolis, IN (US)

(21) Appl. No.: 15/590,309

(22) Filed: May 9, 2017

(51) Int. Cl. A61F 13/505; A61F 13/49

(52) U.S. Cl. CPC A61F 13/505; A61F 13/49061; A61F 13/49011; A61F 2013/15146; A61F 13/49009; A61F 13/49058; A61F 13/49001

(57) ABSTRACT

A disposable absorbent undergarment assembly includes briefs that include a front panel and a rear panel attached along lateral edges and a bottom edge to form a waist opening and a pair of leg openings. Each of the lateral edges has a break therein extending through each of the waist opening an adjacent one of the leg openings. Closures are used for selectively opening or closing each break. An absorbent area is defined in a central area of the front and rear panels. An elastic band forms a continuous loop that is free of any breaks. The elastic band has an upper edge and a lower edge. A first mating member is attached to the lower edge and a second mating member is attached to the briefs adjacent to the waist opening. The first and second mating members releasably attach the elastic band to the briefs.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIE THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

### (2) Description of Related Art Including Information Disclosed Under 37 CFR 1.97 and 1.98

The disclosure and prior art relates to absorbent undergarment devices and more particularly pertains to a new absorbent undergarment device for allowing a person to remove and replace soiled undergarments without the need to remove their pants.

### (h) BRIEF SUMMARY OF THE INVENTION

An embodiment of the disclosure meets the needs presented above by generally comprising briefs configured to be worn by a person. The briefs include a front panel and a rear panel attached along lateral edges and a bottom edge to form a waist opening and a pair of leg openings. Each of the lateral edges has a break therein extending through each of the waist opening an adjacent one of the leg openings. The lateral edges include a closure for selectively opening or closing the break. An absorbent area is defined in a central area of the front and rear panels and at the bottom edge. The absorbent area is configured to absorb and retain fluids. An elastic band forms a continuous loop that is free of any breaks. The elastic band has an upper edge and a lower edge. A first mating member is attached to the lower edge and a second mating member is attached to the briefs adjacent to the waist opening. The first and second mating members are releasably attached to each other to releasably secure the elastic band to the briefs. The briefs are configured to be removed from the first mating member such that the briefs are removable from a person's body while the elastic band remains on the body.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a disposable absorbent undergarment assembly according to an embodiment of the disclosure.

FIG. 2 is a front view of an embodiment of the disclosure.

FIG. 3 is a rear view of an embodiment of the disclosure.

FIG. 4 is a rear view of an embodiment of the disclosure.

FIG. 5 is a front in-use view of an embodiment of the disclosure.

### (j) DETAILED DESCRIPTION OF THE INVENTION

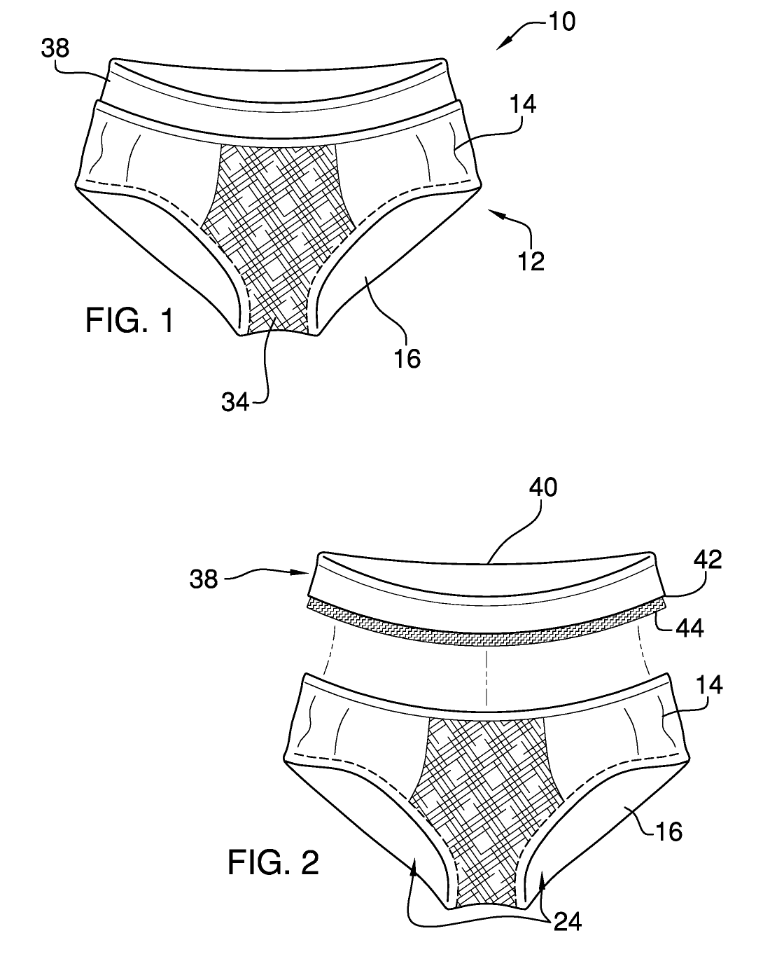

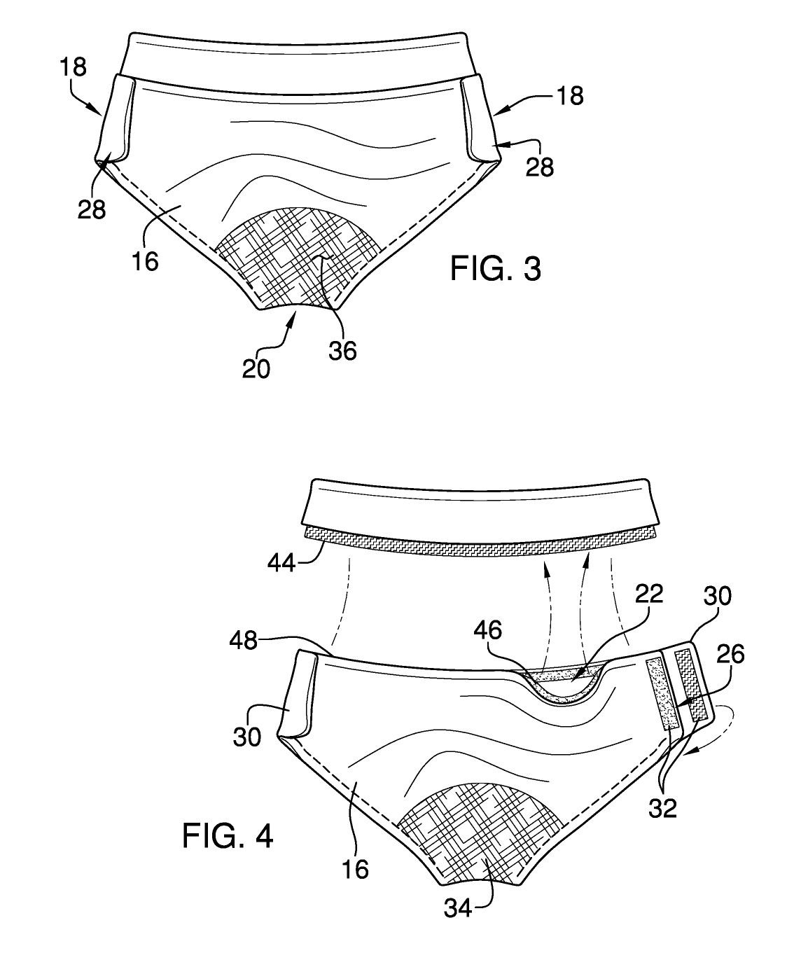

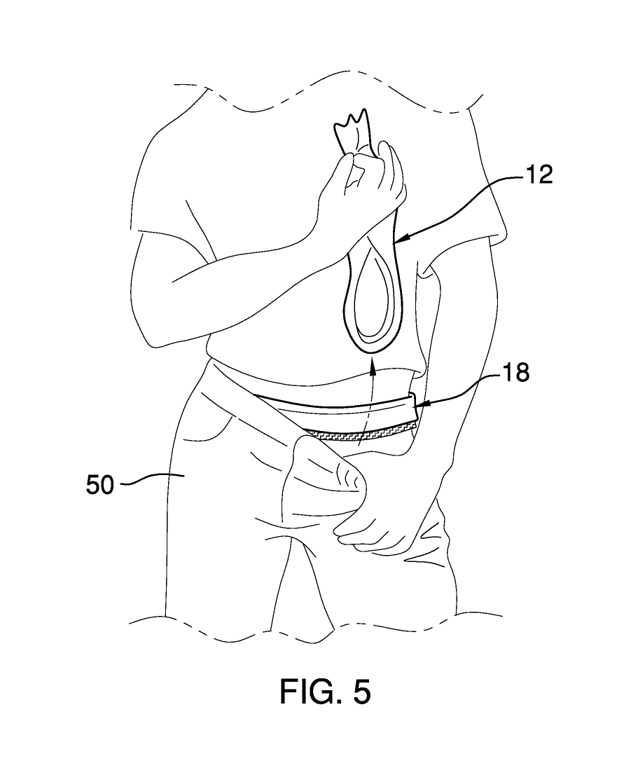

With reference now to the drawings, and in particular to FIGS. 1 through 5 thereof, a new absorbent undergarment device embodying the principles and concepts of an embodiment of the disclosure and generally designated by the reference numeral 10 will be described.

As best illustrated in FIGS. 1 through 5, the disposable absorbent undergarment assembly 10 generally comprises briefs 12 that are configured to be worn by a person. The briefs 12 include a front panel 14 and a rear panel 16 that is attached along shared lateral edges 18 and a bottom edge 20 to form a waist opening 22 and a pair of leg openings 24. Each of the lateral edges 18 has a break 26 therein extending through each of the waist opening 22 an adjacent one of the leg openings 24. Alternatively, the breaks 26 may instead comprise frangible edges such as perforated edges that may be easily broken though full breaks 26 are preferred. Each of the lateral edges 18 includes a closure 28 for selectively opening or closing the associated break 26. The closure 28 may include a flap 30 and a fastener 32 wherein the fastener 32 includes a hook and loop fastener having a first portion on the flap 30 and a second portion on the briefs 12. The flaps 30 may be attached to the front panel 14 as shown in FIG. 4 such that the second portion of the hook and loop fastener is positioned on the rear panel 16.

An absorbent area 34 is defined in a central area of the front 14 and rear 16 panels and at the bottom edge 20. The absorbent area 34 is configured to absorb and retain fluids in a conventional manner with respect to absorbent undergarments. The absorbent area 34 may include an outer surface 36 comprised of a plastic or elastomeric material which is fluid impermeable. An inner surface would be permeable to retain the fluids within the briefs 12 and prevent their bleeding outwardly to the person's clothing.

An elastic band 38 forms a continuous loop, which, unlike the briefs 12, is free of any breaks 26. The elastic band 38 has an upper edge 40 and a lower edge 42. A first mating member 44 is attached to the lower edge 42 and a second mating member 46 is attached to the briefs 12 adjacent to the waist opening 22; that is a top edge 48 of the briefs 12. The first 44 and second 46 mating members are releasably attached to each other to releasably secure the elastic band 38 to the briefs 12. The first mating member 44 is continuous with the lower edge 42 and the second mating member 46 is continuous with the top edge 48 of the briefs 12. The first 44 and second 46 mating members define a hook and loop fastener.

In use, the briefs 12 are worn in a conventional manner as an undergarment complete with the elastic band 38. However, should the briefs 12 be soiled, the briefs 12 are configured to be removed from the second mating member 46 and the breaks 26 opened so that the briefs 12 are removable from a person's body while the elastic band 38 remains on the body. A new pair of briefs 12 is then placed on the body and attached to the elastic band 38 without the need to remove outer coverings such as pants 50.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An undergarment assembly comprising:
briefs being configured to be worn by a person, said briefs including a front panel and a rear panel being attached along lateral edges and a bottom edge to form a waist opening and a pair of leg openings, each of said lateral edges having a break therein extending through each of said waist opening an adjacent one of said leg openings, each of said lateral edges including a closure for selectively opening or closing said break;
an absorbent area being defined in a central area of said front and rear panels and at said bottom edge, said absorbent area being configured to absorb and retain fluids;
an elastic band forming a continuous loop and being free of any breaks, said elastic band having an upper edge and a lower edge;
a first mating member being attached to said lower edge, a second mating member being attached to said briefs adjacent to said waist opening, said first and second mating members being releasably attached to each other to releasably secure said elastic band to said briefs; and
and wherein said briefs are configured to be removed from said first mating member such that said briefs are removable from a person's body while said elastic band remains on the body.

2. The undergarment assembly according to claim 1, wherein said closure includes a flap and a fastener.

3. The undergarment assembly according to claim 1, wherein said absorbent area has an outer surface being fluid impermeable.

4. The undergarment assembly according to claim 1, wherein said first mating member is continuous with said top edge and said second mating member is continuous with a top edge of said briefs, said first and second mating members defining a hook and loop fastener.

5. An undergarment assembly comprising:
briefs being configured to be worn by a person, said briefs including a front panel and a rear panel being attached along lateral edges and a bottom edge to form a waist opening and a pair of leg openings, each of said lateral edges having a break therein extending through each of said waist opening an adjacent one of said leg openings, each of said lateral edges including a closure for selectively opening or closing said break, said closure including a flap and a fastener;
an absorbent area being defined in a central area of said front and rear panels and at said bottom edge, said absorbent area being configured to absorb and retain fluids, said absorbent area having an outer surface being fluid impermeable;
an elastic band forming a continuous loop and being free of any breaks, said elastic band having an upper edge and a lower edge;
a first mating member being attached to said lower edge, a second mating member being attached to said briefs adjacent to said waist opening, said first and second mating members being releasably attached to each other to releasably secure said elastic band to said briefs, said first mating member being continuous with said top edge and said second mating member being continuous with a top edge of said briefs, said first and second mating members defining a hook and loop fastener; and
and wherein said briefs are configured to be removed from said first mating member such that said briefs are removable from a person's body while said elastic band remains on the body.
